(19) United States
(12) Patent Application Publication
(10) Pub. No.: US 2003/0212013 A1
(43) Pub. Date: Nov. 13, 2003

(54) Use of amp kinase activators for treatment type 2 diabetes and insulin resistance

(76) Inventor: William W Winder, Provo, UT (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/297,114

(22) PCT Filed: Jun. 14, 2001
(86) PCT No.: PCT/US01/19283

(51) Int. Cl. A61K 31/7056; A61K 31/7076

(52) U.S. Cl. 514/43; 514/47

(57) ABSTRACT

A method of treating type 2 diabetes in a mammal is provided. The method includes the step of administering a therapeutically effective amount of an AMP-activated protein kinase activator to the mammal. The mammal may be for example, a human, a rat, a mouse, and the like. The AMP-activated protein kinase activator can be subcutaneously injected into the mammal or administered in any other manner that provides for uptake of the AMP-activated protein kinase activator into the cells of the mammal. The activation of the AMP-activated protein kinase activator can produce the benefits of exercise training including the translocation of GLUT4 in the muscle cells of the mammal. A method of treating insulin resistance in a mammal is also provided. To treat the insulin resistance a therapeutically effective amount of an AMP-activated protein kinase activator is given to the mammal.

### 1. RELATED APPLICATIONS

This application is related to and claims the benefit of U.S. Provisional Application Serial No. 60/212,476 of William W. Winder filed Jun. 16, 2000 and entitled “Use of AMP Kinase Activators for Treatment of Type 2 Diabetes,” which is incorporated herein by this reference.

### 2. FIELD OF THE INVENTION

The present invention relates to the methods of treatment of type 2 diabetes and insulin resistance. More specifically, the invention relates to methods of treatment of type 2 diabetes and insulin resistance through artificial activation of AMP kinase.

### 3. TECHNICAL BACKGROUND

Type 2 diabetes is characterized by relative insensitivity to the actions of insulin on glucose uptake. American Diabetes Association: Report of the expert committee on the diagnosis and classification of diabetes mellitus. Diabetes 1998; 21:S5-S19, 1998; Ferrannini, E Endocr Rev 19:477-490 (1997); Gerich, J E Endocr Rev 19:491-503 (1997). In the early stages of this disease, increased insulin secretion can compensate for the insensitivity, but in later stages, insulin deficiency can occur resulting in marked hyperglycemia. Patients with Type 2 diabetes also have dyslipidemia and increased hepatic glucose production. In order to understand the insulin-insensitivity, it is important to understand the basic mechanisms for glucose uptake into the muscle cell, since skeletal muscle represents a large proportion of the insulin-sensitive tissue in the body.

The skeletal muscle sarcolemma and its transverse-tubule (T-tubule) extensions into the muscle fiber interior allow glucose entry into the muscle sarcoplasm via glucose transporters. Holloszy, J O & Hansen, P A Rev Physiol Biochem Pharmacol 128:99-193 (1996); Hayashi, T et al. Am J Physiol 273:E1039-E1051 (1997); Goodyear, L J & Kahn B B: Annu Rev Med 49:235-261 (1998). Each of these transporters consists of a protein that forms a selective hydrophilic passageway through the phospholipid bilayer of the sarcolemma (plasma membrane of muscle), which is a barrier to entrance of water soluble molecules to the interior of the muscle fiber.

There are several kinds of glucose transporters, only one of which is controlled by insulin GLUT4, the insulin sensitive glucose transporter, is present in the muscle fiber in two locations: inserted into the membranes of the sarcolemma and T-tubules, and inserted into the membrane of microvesicles in the sarcoplasm. In the absence of insulin stimulation, the majority of these transporters are located in the microvesicles in the sarcoplasm. When insulin binds to its receptor, tyrosine kinase of the intracellular domain of the receptor is activated, resulting in phosphorylation of insulin receptor substrate-1(IRS-1). The phosphorylated tyrosine residues of IRS-1 (a protein) then serve as docking/activation sites to activate other proteins in the signaling pathway, including phosphatidyl-inositol-3-kinase (PI3K). Activation of PI3K then triggers by undefined mechanisms, the translocation of GLUT4 from the microvesicle fraction to the membrane fraction (sarcolemma and T-tubules). Pessin, J E et al. J Biol Chem 274:2593-2596 (1999). The increased numbers of GLUT4 in the surface membranes provide additional passageways for entrance of glucose into the interior of the muscle fiber.

More recently another signaling pathway was identified which allows GLUT4 translocation and enhancement of glucose uptake into muscle even in the absence of insulin. Holloszy, J O & Hansen, P A Rev Physiol Biochem Pharmacol 128:99-193 (1996); Hayashi, T et al. Am J Physiol 273:E1039-E1051 (1997); Goodyear, L J & Kahn B B: Annu Rev Med 49:235-261 (1998). Muscle contraction triggers movement of GLUT4 from the microvesicle fraction to membranes of the T-Tubules and sarcolemma. As with insulin, the detailed mechanisms of contraction-stimulated GLUT4 translocation and glucose uptake have not been well-defined, but recent data have implicated a new signaling pathway for this process which does not involve the insulin receptor, IRS-1, or PI3K. Merrill, G F et al. Am J Physiol 273:E1107-E1112 (1997); Hayashi, T et al. Diabetes 47:1369-1373 (1998); Winder, W W & Hardie, D G Am J Physiol 277:E1-E10 (1999). The function of this second pathway for stimulation of glucose uptake is presumably to allow increased glucose uptake at times of increased energy need, that is, when the muscle contracts.

The sensitivity of muscle to the action of insulin is controlled in part by the extent of chronic exposure of the muscle to exercise. Holloszy, J O & Hansen, P A Rev Physiol Biochem Pharmacol 128:99-193 (1996); Hayashi, T et al. Am J Physiol 273:E1039-E1051 (1997); Goodyear, L J & Kahn B B: Annu Rev Med 49:235-261 (1998); Ivy, J L, Sports Med 24:321-336 (1997); Wallberg-Henriksson, H et al. Sports Med 25:25-35 (1998). Insulin insensitivity develops during several days of bed rest. Mikines, K J, et al. J Appl Physiol 70:1245-1254 (1991). It has been proposed that the reduced insulin-sensitivity that occurs with aging is due to inactivity of muscle.

When muscle is subjected to periods of contraction several days in succession, as would occur during the process of endurance training, the muscle becomes more sensitive to the action of insulin. An increase in glucose uptake continues in the post-exercise period, resulting in glycogen supercompensation. With several days of chronic exercise, an increase in total GLUT4 occurs in both human subjects and in rats. Gulve, E A & Spina, R J et al. J Appl Physiol 79:1562-1566 (1995). For example, 10 days of exercising 2 hr/day at approximately 70% of maximal oxygen consumption was reported to increase total GLUT4 in muscle biopsies by 98%. Gulve, E A & Spina, R J et al. J Appl Physiol 79:1562-1566 (1995). Studies in rats indicate that the doubling of GLUT4 in response to swimming for 6 hrs, five days in succession, is reversed during a 40 hr period of rest. Host, H H et al. J Appl Physiol 84:798-802 (1998). The positive effect of exercise on total GLUT4 appears to require regular exposure of the muscle to exercise. The increase in total GLUT4, measured by Western blot, is preceded by an increase in GLUT4 mRNA. Neufer, P D et al. Dohm G L Am J Physiol 265:C1597-C1603 (1993); Ren, J M et al. J Biol Chem 269:14396-14401 (1997).

A so called, exercise response element on the promoter region of the GLUT4 gene is thought to mediate the effect of exercise on the rate of transcription. Ezaki, O Biochem Biophys Res Commun 241:16 (1997); Tsunoda, N et al. Biochem Biophys Res Commun 267:744-751 (2000). To date, the nature and regulation of the putative transcription factor that binds to this region of the gene and upregulates transcription in response to chronic muscle contraction is unknown Muscle contraction therefore has an acute effect on glucose uptake mediated by an increase in GLUT4 translocation. It also has a more prolonged effect on glucose uptake, possibly due to the depletion of glycogen and enhancement of synthesis of GLUT4 and other intracellular insulin signaling proteins. Kim, Y B et al. Biochem Biophys Res Commun 254:720-727 (1999); Chibalin, A V et al. Proc Natl Acad Sci 97:38-43 (2000).

In persons with insulin resistance and in patients with mild Type 2 diabetes, the responses of both plasma glucose and plasma insulin are markedly exaggerated compared to the normal patient during the three hour period following ingestion of 100 grams of glucose. Much more insulin is required to dispose of the same amount of glucose. When patients with either impaired glucose tolerance or mild Type 2 diabetes are trained by running 40-60 minutes per day, five days/week for 12 months, their insulin and glucose responses to the glucose tolerance test were normalized or dramatically improved. Holloszy, J O et al. Acta Med Scand Suppl 711:55-65 (1986). Less pronounced effects have been reported for mild exercise programs. US Department of Health and Human Services: The effects of physical activity on health and disease. In: Physical Activity and Health: A Report of the Surgeon General. Washington, D.C.: Centers for Disease Prevention and Control, 1996.

Epidemiological evidence indicates that those with more active lifestyles are less prone to develop Type 2 diabetes. One study reported a 6% decrease in incidence of diabetes in male college alumni for each 500 kcal increment in weekly exercise. Helmrich, S P et al. New England J Med 325:147-152 (1991). A recent report indicates that patients with Type 2 diabetes respond to an acute bout of exercise with increased glucose uptake and normal GLUT4 translocation Kennedy, J W et al. Diabetes 48:1192-1197 (1999). Patients with type 2 diabetes appear to have normal quantities of GLUT4, but the defect appears to be in ability to translocate the GLUT4 to the sarcolemma in response to insulin Handberg, A, et al. Diabetologia 33:625-627 (1990); Pedersen, O et al. Diabetes 39:865-870 (1990); Garvey, W T et al. Diabetes 41:465-475 (1992); Garvey, W T et al. J Clin Invest 101:2377-2386 (1998).

A study by Entgen, et al on fatty Zucker (ZDF) rat, one a model of Type 2 diabetes, showed that the insulin-insensitivity of the fast-twitch types of skeletal muscle could be reversed by exposure of the rats to just two weeks of running on the treadmill, 1 hr/day. Etgen, G J et al. Am J Physiol 272:E864-E869 (1997). The epitrochlearis muscle is a very thin foreleg muscle that can be used for in vitro measurement of uptake of radiolabeled glucose analogs such as 3-O-methyl-glucose (3 MG). Insulin-induced uptake of 3 MG and translocation of GLUT4 to the surface membrane in the epitrochlearis muscle from the fatty Zucker rat is markedly attenuated compared to normal rats. The epitrochlearis from the endurance trained rats showed an approximate doubling of GLUT4. Glucose transport was normalized in epitrochlearis muscles of fatty Zucker rats that were trained on the treadmill for two weeks. As with human diabetic patients, the ZDF rats were not deficient in GLUT4, but insulin fails to trigger sufficient translocation/activation to allow normal glucose transport. Although the mechanism of how training induced increased expression of GLUT4 in ZDF muscle compensates for the deficiency in insulin-stimulated glucose transport is not well-defined, these studies provide evidence that the contraction-induced pathway may indeed be useful for treatment of type 2 diabetes.

In light of the foregoing, it would be an advancement in the art to provide a method of treating type 2 diabetes and insulin resistance that artificially stimulate the response seen in exercise training. It would be a further advancement to provide a method that artificially stimulates GLUT4 translocation. It would be a further advancement to provide a method that could mimic exercise training for an extended period of time. Such methods are disclosed and claimed herein.

### 4. BRIEF SUMMARY OF THE INVENTION

The invention relates to a method of treating type 2 diabetes in a mammal. The method includes the step of administering a therapeutically effective amount of an AMP-activated protein kinase activator to the mammal. The mammal may be for example, a human, a rat, a mouse, and the like. The AMP-activated protein kinase activator can be subcutaneously injected into the mammal or administered in any other manner that provides for uptake of the AMP-activated protein kinase activator into the cells of the mammal. The activation of the AMP-activated protein kinase activator can produce the benefits of exercise training including the translocation of GLUT4 in the muscle cells of the mammal.

The invention also relates to a method of treating insulin resistance in a mammal suffering from obesity, type 2 diabetes, or muscle paralysis. To reduce the insulin resistance a therapeutically effective amount of an AMP-activated protein kinase activator is given to the mammal.

AMP-activated protein kinase can be activated allosterically by increases in the concentration of AMP or by a compound that is analogous to AMP. In one aspect of the invention an AMP analog is administered to a subject so that the AMP analog is taken into the muscle cells of the subject. This may require modification of the analog so that it may be transported into the cell. For example the AMP analog may be adenosine-5′-thimonophosphate, adenosine 5′-phosphoramidate, formycin A 5′-monophosphate, or ZMP Because these AMP analogs are not readily transported into a cell the analog may be administered intracellularly.

5-aminoimidazole-4-carboxamide ribonucleoside (AICAR) is an AMP analog that is phosphorylated in muscle cells to become ZMP. This allows the 5-aminoimidazole-4-carboxamide to enter the cells and then be converted to ZMP to mimic the effect of AMP in the cell. 5-aminoimidazole-4-carboxamide ribonucleoside can be administered at a dose from about 0.5 to at least about 1.0 mg/g body weight.

In one aspect of the invention, the AMP-activated protein kinase activator is administered acutely in a single dose. Such acute administration will result in the activation of AMP kinase for a relatively short period of time. Because the majority of the benefit of exercise in patients suffering from type 2 diabetes or insulin resistance is seen after an extended period of exercise training, the AMP-activated protein kinase activator can be administered chronically. Chronic administration of the AMP anolog refers to the administration of one or more doses daily of an AMP analog for two or more days. For example, one or more daily doses of an AMP analog for a period of weeks has been shown to provide an additional benefit to the subject. To better mimic the effect of exercise training the AMP-activated protein kinase activator can be administered intermittently for a period of time.

### 5. SUMMARY OF THE DRAWINGS

A more particular description of the invention briefly described above will be rendered by reference to the appended drawings and graphs. These drawings and graphs only provide information concerning typical embodiments of the invention and are not therefore to be considered limiting of its scope.

FIG. 1 is a set of bar graphs illustrating AMPK activity in the muscles of rats following an injection with AICAR or a saline control.

FIG. 2 is a graph showing citrate dependence of acetyl-CoA carboxylase in the muscles of rats injected with AICAR or a saline control.

FIG. 3 illustrates the Western blot analysis of GLUT4 protein in muscles of rats injected with AICAR or a saline control for 5 days.

FIG. 4 is a set of bar graphs illustrating relative GLUT4 levels in the muscles of rats treated with AICAR or a saline control for 5 days.

FIG. 5 is a set of bar graphs illustrating the effect on hexokinase activity in muscles of rats injected with AICAR or a saline control for 5 days.

### 6. DETAILED DESCRIPTION OF THE INVENTION

The invention relates to a method of treating type 2 diabetes in a mammal. The method of the present invention may also be used to treat insulin resistance in a mammal suffering from obesisty, type 2 diabetes, or muscle paralysis.

The method includes the step of administering a therapeutically effective amount of an AMP-activated protein kinase activator to the mammal. The term therapeutically effective amount as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated. In other words, therapeutically effective amount is intended to mean an amount of a compound sufficient to produce the desired pharmacological effect. It is understood that the therapeutically effective amount to be used in the treatment of type 2 diabetes or insulin resistance must be subjectively determined according to the type of mammal and the desired effect. Variables involved include the size of the patient, the type of AMPK activator, the state of the disease, age of the patient, and response pattern of the patient. The novel methods of the invention for treating, preventing or alleviating the conditions described herein, comprise administering to mammals in need thereof, including humans, an effective amount of one or more compounds of this invention or a non-toxic, pharmaceutically acceptable addition salt thereof. The compounds may be administered subcutaneously, orally, rectally, parenterally, or topically to the ski and mucosa. Moreover, because many of the known AMP analogs are phosphorylated, it is difficult to get an effective amount of the analog inside a cell by injection or topical methods. Thus, it maybe necessary to administer the analog directly into the muscle of the mammal by for example methods of in vivo electroporation.

The mammal may be for example, a human, a rat, a mouse, and the like. The AMP-activated protein kinase activator can be subcutaneously injected into the mammal or administered in any other manner that provides for uptake of the AMP-activated protein kinase activator into the cells of the mammal. The activation of the AMP-activated protein kinase activator can produce the benefits of exercise training including the loss of body fat.

AMP-activated protein kinase can be activated allosterically by increases in the concentration of AMP or by a compound that is analogous to AMP. In one aspect of the invention an AMP analog such as adenosine-5′-thiomonophosphate, adenosine 5′-phosphoramidate, formycin A 5′-monophosphate, or ZMP is administered to a subject so that the AMP analog is taken into the cells of the subject. This may require modification of the analog so that it may be transported into the cell. Because these AMP analogs are not readily transported into a cell the analog may be administered intracellularly.

5-aminoimidazole-4-carboxamide ribonucleoside (AICAR) is an AMP analog that is phosphorylated in muscle cells to become ZMP. This allows the 5-aminoimidazole-4-carboxamide to enter the cells and then be converted to ZMP to mimic the effect of AMP in the cell. 5-aminoimidazole-4-carboxamide ribonucleoside can be administered at a dose from about 0.5 to at least about 1.0 mg/g body weight. It has been shown that an effective dose in a rat is about 1.0 mg/g body weight. However, when determining the dose the treatment of a human, the dose may be higher or lower.

In one aspect of the invention, the AMP-activated protein kinase activator is administered acutely in a single dose. This provides the acute activation of AMPK and provides for a short-lived effect similar to a single bout of exercise. However, the AMP-activated protein kinase activator can be administered chronically over a period days or weeks to provide an additional benefit to the subject. Providing a dose for a chronic period of about 28 days has been shown to give significant benefits over a single acute activation of AMPK.

The AMPK activator can also be administered intermittently over a period of time to better mimic the effect of exercise training. Such intermittent activation can consist of activating AMPK for a period of at least one day, followed by a period of non-activation for at least one day, followed by an additional period of activation of at least one day. The period of activation followed by non-activation can be repeated as needed to obtain the desired results. For example an increased effectiveness was observed when rats were intermittently injected with AICAR as follows: injection for 3 days, followed by 2 days without injection, followed by 5 days of injection, followed by two days without injection, followed by 3 days with injection.

Numerous studies have demonstrated beneficial effects of regular endurance exercise in increasing insulin-sensitivity of muscle. Goodyear, L. J., & B. B. Kahn. Annu. Rev. Med. 49:235-261 (1998); Hayashi, T. et al. Am. J. Physiol. 273 (36):E1039-E1051 (1997); Holloszy, J. O. & P. A. Hansen 128:99-193 (1996); Ivy, J. L. Sports Med. 24:321-336 (1997); Wallberg-Henriksson, H., J. et al. Sports Med. 25:25-35 (1998). In both animals and humans, a few bouts of exercise will increase total GLUT4 and increase insulin sensitivity. Gulve, E. A, & R. J. Spina. J. Appl. Physiol. 79:1562-1566 (1995). Patients with type 2 diabetes do not have a deficiency in total GLUT4 in muscle, but insulin-induced translocation of GLUT4 to the cell surface is defective. Garvey, W. T., et al. Diabetes 41:465-475 (1992); Handberg, A. et al. Diabetologia 33:625-627 (1990); Pedersen, O. et al. Diabetes 39:865-870 (1990); Garvey, W. T., et al. J. Clin. Invest. 101:2377-2386 (1998); Kelley, D. E. et al. J. Clin. Invest. 97:2705-2713 (1996).

Etgen, et al demonstrated that two weeks of exercise, 1 hour per day, would increase total GLUT4 and compensate for this defect in the fatty Zucker rat, an animal model of type 2 diabetes. Etgen, G. J., et al. Am. J. Physiol. 272:E864-E869 (1997). They postulated that this adaptation to training increased insulin-recruitable GLUT4 in the muscle fibers, thereby allowing increased glucose transport in response to insulin. It is the chronic contraction-induced activation of AMPK that triggers the increase in GLUT4. Thus, chemical stimulation of AMPK can be used to increase GLUT4 and to treat insulin resistance and type 2 diabetes.

GLUT4 mRNA is increased in muscle in response to endurance exercise training. The GLUT4 gene has what has been termed an exercise response element residing between 442 and 1000 base pairs upstream from the transcription start site. Ezaki, O. Biochem. Biophys. Res. Comm. 241:1-6(1997); Tsunoda, N. et al. Biochem. Biophys. Res. Comm. 239:503-509(1997). Nuclear run-on analyses have clearly demonstrated an increase in muscle GLUT4 mRNA synthesis with training. Neufer, P. D. & G. L. Dohm. Am. J. Physiol. 265:C1597-C1603 (1993). In animals that had been exercising for several days, an increase in GLUT4 message was observed three hours following a bout of exercise. An increase in GLUT4 gene transcription was not observed in non-trained rats after a single bout of exercise, implying that other mechanisms besides regulation of transcription may be operative in causing the initial increase in GLUT4. Others have reported an increase in GLUT4 mRNA in response to a single bout of exercise. Ren, J. M. et al. J. Biol. Chem. 269:14396-14401(1994).

Data provided herein suggest the possibility that what has been termed the exercise response element of the GLUT4 gene may actually be an AMPK response element. GLUT4 and hexokinase gene expression increases in response to AMPK activation. Transcription factors binding to that region of the gene can be screened to see if they have target sites for phosphorylation by AMPK. Previous studies provide evidence of regulation of transcription of hepatocyte genes by AMPK. Treatment of isolated hepatocytes with AICAR has been shown to decrease pyruvate kinase and fatty acid synthetase gene expression.

AMPK activation will increase glucose uptake. AMPK is shown herein to be chemically activated by AICAR, thus mimicking the effect of muscle contraction. AICAR also induces the translocation of the GLUT4 transporter to the membrane surface of the muscle, thus allowing an increase in glucose uptake. See Kurth-Kraczek et al. Diabetes 48:1667-1671 (1999).

One of the defects of type 2 diabetes is that the muscle becomes less sensitive to insulin. This defect appears to be a deficiency in the amount of GLUT4 translocated to the cell surface in response to insulin. Treatment with AICAR results in an increase of the total GLUT4 and hexokinase in the muscle. This is also a benefit to patients with type 2 diabetes.

In one animal model of type 2 diabetes, an increase in total GLUT4 induced by daily bouts of treadmill running was shown to compensate for a deficiency in capacity to translocate GLUT4 to the cell surface in response to insulin. See Entgen et al., Am. J. Physiol. 272:E864-E869 (1997). Human muscle contains AMPK which was activated in response to muscle contraction. Thus, AICAR or other AMPK can activators can be used to treat patients with type 2 diabetes.

Other analogues of 5′-AMP have been found to activate AMPK more potently in vitro including adenosine-5′-thiomonophosphate and adenosine 5′-phosphoramidate. Formycin A 5′-monophosphate and ZMP activate less potently. See Hardie & Carling, Eur. J. Biochem. 246:259-273 (1997). AICA-riboside is the only adenosine analog that has been found useful in activating AMPK in vivo and which has been utilized to show effects of chronic activation of this kinase.

In order to better describe the details of the present invention, the following discussion is divided into four sections: (1) exercise has an acute insulin-like effect; (2) actions of AMP-activated protein kinase; (3) AMPK can be artificially activated; and (4) AMPK mediates the effect of muscle exposure to exercise.

6.1 Exercise has an Acute Insulin-Like Effect

A considerable amount of data has accumulated showing that contraction of muscle has an acute insulin-like effect, triggering the uptake of glucose. Chronic muscle contraction, as seen in endurance training has effects on insulin sensitivity, enhancing the effect of insulin on glucose uptake. Endurance training results in an increase in levels of GLUT4 in the muscle. This increase in GLUT4 is thought to be responsible in part for the enhancement of insulin sensitivity. Thus, exercise has been used as a treatment for patients suffering from type 2 diabetes and insulin insensitivity. Both the acute and chronic effects of muscle contraction on glucose uptake and the increase in GLUT4 are to activation of a protein kinase, AMP-activated protein kinase (AMPK). This kinase is activated by the increase in 5′-AMP and the decline in creatine phosphate that occur during muscle contraction. Phosphorylated AMPK then presumably phosphorylates undefined target proteins which in turn increase glucose uptake and transcription of the GLUT4 gene.

6.2 Actions of AMP-Activated Protein Kinase

AMP-activated protein kinase (AMPK) was discovered at approximately the same time as cAMP-dependent protein kinase, but only recently have important regulatory functions relating to diabetes been elucidated. Winder, W W & Hardie, D G Am J Physiol 277:E1-E10 (1999). It consists of three subunits (α, β, and γ) and for each subunit there are at least two different isoforms. Hardie, D G & Carling, D Eur J Biochem 246:259-273 (1997); Hardie, D G et al. Ann Rev Biochem 67:821-855 (1998). This kinase is activated by both phosphorylation and allosteric mechanisms. It can be phosphorylated and activated by an upstream kinase, AMPKK, and is also activated allosterically by increases in the 5′-AMP/ATP ratio. In muscle, creatine phosphate (CP) allosterically inhibits the enzyme. Ponticos, M et al. EMBO J 17:1688-1699 (1998). In liver, AMPK plays the role of phosphorylating and inactivating acetyl-CoA carboxylase (ACC) and 3-hydroxy-3-methylglutaryl-CoA reductase (HMGR), the rate limiting enzymes of fatty acid and cholesterol biosynthesis.

AMPK is activated in skeletal muscle of rats during treadmill running and in response to electrical stimulation. Winder, W W & Hardie, D G Am J Physiol 270:E299-E304 (1996); Hutber, C A et al. Am J Physiol 272:E262-E266 (1997); Vavvas, D et al. J Biol Chem 272:13256-13261 (1997); Ihlemann, J et al. Am J Physiol 277:E208-E214 (1999); Rasmussen, B B & Winder, W W J Appl Physiol 83:1104-1109 (1997). As muscle contracts, ATP is used as a source of energy, generating ADP and inorganic phosphate. The ADP can be phosphorylated to form ATP in the glycolytic pathway in the sarcoplasm or by oxidative phosphorylation in the mitochondria. Non-oxidative, rapidly acting mechanisms also include the action of myokinase, which makes one ATP and one 5′-AMP from two ADP molecules, and the action of creatine phosphokinase, which transfers a phosphate from CP to ADP forming ATP (FIG. 2). These changes occur rapidly at the beginning of muscle contraction resulting in a drop in CP, the allosteric inhibitor of AMPK, and an increase in 5′-AMP, the allosteric activator of AMPKK and AMPK.

Skeletal muscle is not a lipogenic tissue, but it was found to contain a unique isoform of ACC. Winder, W W & Hardie, D G Am J Physiol 277:E1-E10 (1999). The malonyl-CoA synthesized by ACC in skeletal muscle is important in control of fatty acid oxidation. Malonyl-CoA is a potent inhibitor of carnitine palmitoyl-transferase 1 (CPT 1), the rate limiting step in transfer of long-chain fatty acyl-CoA into the mitochondria where oxidation can occur. McGarry, J D & Brown, N F Eur J Biochem 244:1-14 (1997). In rats running on the treadmill AMPK was found to be activated in the exercising muscle concurrently with inactivation of ACC and a drop in malonyl-CoA. Winder, W W & Hardie, D G Am J Physiol 270:E299-E304 (1996). The decline in malonyl-CoA was postulated to be important in allowing an increase in fatty acid oxidation as exercise continued.

6.3 AMPK can be Artificially Activated

An analog of adenosine, 5-aminoimidazol-4-carboxamide-riboside (AICAR), can be used to artificially activate AMPK in liver cells, resulting in inactivation of ACC and HMGR. Winder, W W & Hardie, D G Am J Physiol 277:E1-E10 (1999); Hardie, D G & Carling, D Eur J Biochem 246:259-273 (1997); Hardie, D G et al. Ann Rev Biochem 67:821-855 (1998); Henin, N et al. FASEB 9:541-546 (1995). AICAR is taken up by cells and phosphorylated to form the corresponding monophosphorylated nucleotide (ZMP), an analog of 5′-AMP. Perfusion of rat hindlimb muscle with AICAR resulted in accumulation of ZMP, activation of AMPK with consequent inactivation of ACC, a decrease in malonyl-CoA, and an increase in palmitate oxidation. Merrill, G F et al. Am J Physiol 273:E1107-E1112 (1997). Surprisingly, along with the increase in fatty acid oxidation was observed an increase in glucose uptake by the perfused hindlimb.

The activation of AMPK during muscle contraction leads to activation of fatty acid oxidation and increased glucose uptake to meet the increased energy needs of contracting muscle. The incubation of rat epitrochlearis muscles with AICAR activated AMPK and stimulated an increase in uptake of 3 MG. Hayashi, T et al. Diabetes 47:1369-1373 (1998). The increase in 3 MG uptake caused by AICAR was not blocked by Wortmannin, the inhibitor of PI3K that completely blocks the effect of insulin on glucose uptake. Effects of AICAR were additive with the effects of insulin, but not with contraction. These acute effects of AICAR in isolated muscle are also seen in papillary muscle and in both high and low oxidative muscle types of rats infused with AICAR in vivo. Bergeron, R et al. Am J Physiol 276:E938-E944 (1999); Russell, R R et al. Am J Physiol 277:H643-H649 (1999).

Experiments on isolated epitrochlearis have clearly demonstrated that when AMPK is activated by hypoxia or other perturbations expected to increase AMP and decrease CP, glucose transport is also stimulated. Hayashi, T et al. Diabetes 49:527-531 (2000). Perfusion of rat hindlimbs with AICAR triggers translocation of GLUT4 from the microvesicle fraction of the muscle to the membrane fraction. Kurth-Kraczek, E J et al. Diabetes 48:1667-1671 (1999).

Infusion of AICAR results in a decrease in lipolysis, a suppression of endogenous glucose production, and stimulation of glucose uptake into the white, low oxidative region of the gastrocnemius, but not in soleus of both lean and ZDF rats. Bergeron R, et al. Diabetes 49(suppl 1):A278 (2000). AICAR injection (0.25 mg/g body weight) acutely decreases blood glucose for 4-6 hours in normal mice (C57/BJ6), insulin deficient diabetic mice (C57/BJ6STZ), and in insulin resistant diabetic mice (KKA^{y}). Nakano, M et al. Diabetes 49(suppl 1):A12 (2000). AMPK activation is an important intermediate step, coupling muscle contraction with an increase in GLUT4 translocation and glucose uptake by the muscle.

6.4 AMPK Mediates the Effect of Muscle Exposure to Exercise

When resting rats are injected with AICAR an increase in muscle AMPK activity occurs within 15 minutes and continues for at least two hours. When rats are injected in this manner for 5 days in succession, an increase in GLUT4, hexokinase activity, and glycogen content of the muscle occurs, similarly to the adaptations seen in response to endurance exercise training. Holmes, B F et al. J Appl Physiol 87:1990-1995 (1999). The increase in GLUT4 and hexokinase were found to continue for at least 4 weeks with daily injections of AICAR. Winder, W W et al. J Appl Physiol 88:2219-2226(2000). This effect of artificial stimulation of AMPK with AICAR on GLUT4 expression has also been confirmed in isolated epitrochlearis muscle exposed to AICAR for 18 hrs. Ojuka, E O et al. J Appl Physiol 88:1072-1075 (2000). Insulin deficient diabetic and insulin insensitive diabetic mice respond to 5-7 days of AICAR injections with an increase in muscle GLUT4 content. These studies suggest that it is the chronic AMPK activation occurring with each bout of training that mediates the stimulation of GLUT4 expression. They also demonstrate the possibility of employing pharmaceutical activators of AMPK in treating insulin insensitivity and type 2 diabetes.

### 7. EXAMPLES

The following examples are given to illustrate various embodiments which have been made with the present invention. It is to be understood that the following examples are not comprehensive or exhaustive of the many types of embodiments which can be prepared in accordance with the present invention.

### Example 1

Acute Injection of AICAR

All procedures were approved by the Institutional Animal Care and Use Committee. Male Sprague-Dawley rats weighing 197±5 grams (Sasco, Wilmington, Mass. 01887) were housed in individual cages in a temperature (22-25 C.) and light-controlled (12:12-h light-dark cycle) room and were given food (Harlan Teklad rodent diet, Madison, Wis.) and water ad libitum. To determine acute in vivo effects of AICAR, a jugular catheter was installed and exteriorized on the back of the neck three days prior to the day of the experiment. This catheter was implanted for the purpose of allowing rapid anesthesia of the rat and rapid blood and tissue collection. Rats were then given AICAR (1 mg/g body weight) subcutaneously in sterile 0.9% NaCl or were given 0.9% NaCl (n=7 in each group). One hour following the subcutaneous injection of AICAR, rats were anesthetized by intravenous injection of pentobarbital (4.8 mg/100 g body weight). The epitrochlearis and gastrocnemius/plantaris muscles were quickly removed and rapidly frozen with stainless steel clamps at liquid nitrogen temperature. Blood was collected via the abdominal aorta and a perchloric acid extract was prepared (0.5 ml blood to 2.0 ml 10% HClO₄), neutralized and utilized for analysis of glucose and lactate.

Muscles from rats killed 1 hour following injection of AICAR or saline were analyzed for AMPK, citrate-dependence of acetyl-CoA carboxylase, malonyl-CoA, glycogen, ZMP, ZTP, ATP, and ADP. Hassid, W Z. & S. Abraham Methods Enzymol. 3:35-36 (1957); McGarry, J. D. et al. J. Biol. Chem. 253:8291-3293 (1978). Blood glucose and lactate were measured by enzymatic techniques. Bergmeyer, H. U., et al. D-Glucose determination with hexokinase and glucose-phosphate dehydrogenase. In: Methods of Enzymatic Analysis, edited by H. U. Bergmeyer. New York: Academic, 1196-1201 (1974); Gutmann I., & A. W. Wahlefeld. L-(+)-Lactate. Determination with lactate dehydrogenase and NAD. In: Methods of Enzymatic Analysis, edited by H. U. Bergmeyer. New York: Academic, 1464-1468 (1974).

Rats weighing 197±5 grams tolerate injections of 1 mg/g body weight daily of AICAR well with no apparent discomfort. This dose was found to increase ZMP in gastrocnemius muscle from non-detectable values to 0.91±0.02 μmol/g 60 minutes following the injection. This was in the same range as was seen in the preliminary time course experiments. The levels of tissues and blood metabolites of rats sacrificed 60 minutes following an injection are shown in Table 1. ATP and ADP were not influenced by the injection with AICAR. ZTP increased from non-detectable levels to 1.2 μmol/g. Rats injected with AICAR were found to have significantly increased blood lactate (p<0.001) and decreased blood glucose (p<0.01) compared to controls. Muscle glycogen and liver glycogen were not acutely influenced 60 minutes following a single injection of AICAR.

TABLE 1
Tissue and blood metabolites of rats killed 60 minutes following an
injection of saline or AICAR.

| | Saline | AICAR |
|---|---|---|
| | Injected Rats | injected Rats |
| Muscle Glycogen (μmol/g) | 43 ± 2 | 44 ± 4 |
| Muscle ATP (umol/g) | 8.1 ± 0.1 | 7.6 ± 0.1 |
| Muscle ADP (μmol/g) | 0.97 ± 0.01 | 0.97 ± 0.02 |
| Muscle ZMP (μmol/g) | Not Detectable. | 0.91 ± 0.02* |
| Muscle ZTP (μmol/g) | Not Detectable | 1.18 ± 0.09* |
| Muscle Malonyl-CoA | 1.6 ± 0.1 | 0.6 ± 0.1* |
| (nmol/g) | | |
| Blodd Glucose (mM) | 7.4 ± 0.2 | 5.6 ± 0.6* |
| Blood Lactate (mM) | 1.7 ± 0.1 | 6.8 ± 0.5* |
| Liver Glycogen (μmol/g) | 333 ± 16 | 344 ± 44 |

Values are means ± SEM, n = 7.
*Significantly different from saline injected rats, p < 0.01. Muscles are gastrocnemius/plantaris.

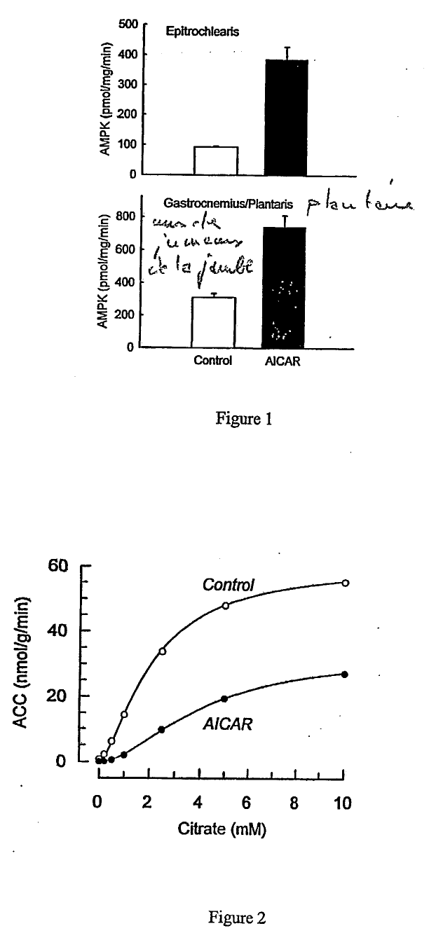

Referring to FIG. 1, the AMPK activity in epitrochlearis and gastrocnemius/plantaris muscles of rats 1 hour following injection with 1 mg/g body weight AICAR or with saline is shown. Values are means ±SEM. The values for AICAR treated rats are significantly different from those of saline treated rats for both epitrochlearis and gastrocnemius/plantaris, p<0.001. AMPK activity was increased 2.4 fold in gastrocnemius/plantaris muscles and 4 fold in the epitrochlearis muscles 1 hour following the AICAR injection.

Referring to FIG. 2, the citrate dependence of acetyl-CoA carboxylase in gastrocnemius/plantaris muscles of rats 1 hour following injection with AICAR (1 mg/g body weight) or saline is illustrated. Standard errors were determined but are not shown. Curves shown were fitted to data using the Hill Equation and Grafit software (Sigma Chemical, St. Louis, Mo.) as described previously. Merrill, G. F. et al. Am. J. Physiol. 273(36):E1107-E1112 (1997). Gastrocnemius/plantaris ACC activity was markedly influenced by AICAR injection. The maximal velocity of the reaction (Vmax) as a function of citrate concentration was reduced from 60.1±1.3 to 32.4±1.3 nmol/g/min (p<0.001). The citrate activation constant (Ka) was increased from 3.1±0.1 to 13.0±0.3 mM (p<0.001). Because of the limited amount of tissue, the entire citrate activation curve could not be determined for epitrochlearis muscle, but the activity of ACC at a physiological concentration of citrate (0.2 mM) was reduced from 0.50±0.10 nmol/g/min to 0.10±0.03 nmol/g/min. Gastrocnemius/plantaris malonyl-CoA was likewise significantly (p<0.001) lower in the AICAR injected rats compared to controls 1 hour following the injection as shown in Table 1.

The acute studies clearly demonstrate that AMPK is activated in epitrochlearis and gastrocnemius/plantaris of rats injected with AICAR. Additional evidence of activation of AMPK is provided by the fact that the kinetic properties of ACC change similarly to what is seen when purified ACC is phosphorylated in vitro. Winder, W. W. & D. G. Hardie, Am. J. Physiol. 270:E299-E304 (1996). ACC is a downstream target protein for AMPK. Phosphorylation of ACC by AMPK during exercise has been postulated to be responsible for decreasing the muscle content of malonyl-CoA, an inhibitor of carnitine palmitoyl-transferase 1 (CPT1) and allowing therefore an increase in oxidation of long chain fatty acids as they become available.

Results from the current study show that the effects normally occurring during exercise can also be triggered in vivo by chemical activation of the AMPK. One of the postulated causes of insulin resistance in the type 2 diabetes is elevated muscle malonyl-CoA which would inhibit fatty acid oxidation and increase long-chain acyl-CoA concentrations in the cells. Ruderman, N B. et al. Am. J. Physiol. 276:E1-E18 (1999). Although no data is currently available on malonyl-CoA in muscle of human diabetic, these studies clearly demonstrate the feasibility of manipulation of malonyl-CoA by drugs designed to activate the AMPK signaling system iii vivo.

The decrease in blood glucose in response to a single injection of AICAR is consistent with either an increase in glucose uptake into peripheral tissues and/or a decrease in glucose production by the liver. The increase in glucose uptake stimulated by AICAR results in increased rates of lactate production in the resting muscle. Kurth-Kraczek, E. J. et al. Diabetes 48 (1999); Merrill, G. F. et al. Am. J. Physiol. 273(36):E1107-E1112(1997). The increase in concentration of blood lactate in the AICAR injected rats is consistent with the idea that glucose uptake is enhanced resulting in increased glycolytic flux. Liver glycogenolysis or glycogen synthesis did not appear to be influenced 60 minutes following an AICAR injection. Hepatic gluconeogenesis may be inhibited at the fructose-1,6-bisphosphatase reaction. Vincent, M. F. et al. Adv. Exp. Med. Biol. 309B:1991-1995 (1991); Vincent, M. F. et al. Diabetologia 39:1148-55 (1996). A reduction in utilization of lactate for glucose production by the liver may also have contributed to the decrease in blood glucose and increase in blood lactate.

### Example 2

Chronic Injection of AICAR

To determine the effect of chronic activation of AMPK, rats were injected (between 8 and 10 am.) subcutaneously with AICAR (1 mg/g body weight) or saline vehicle for five days in succession. This dose was shown in preliminary experiments to increase ZMP levels in the muscle to 0.57±0.06 μmol/g after 15 min, to 0.79±0.06 μmol/g after 60 min, to 0.69±0.06 μmol/g after 90 min, and to 0.60±0.06 μmol/g after 120 minutes (n=3 at each time point). Beginning with the first injection, controls were pair fed with AICAR-injected rats. Saline injected controls ate 17±1 g and AICAR injected rats ate 18±1 g of food during the 24 hour period prior to blood and tissue collection. Rats were anesthetized by intraperitoneal injection of pentobarbital (22-25 hrs following the last AICAR injection) and epitrochlearis, and gastrocnemius/plantaris muscles were collected and frozen as described above. Muscles were kept under liquid nitrogen until analyzed.

Muscles from rats killed 22-25 hours following the fifth AICAR injection were analyzed for glycogen and GLUT4. For GLUT4 measurement muscle was ground to powder under liquid nitrogen. See Etgen, G. J., et al. Am. J. Physiol. 272:E864-E869 (1997). A homogenate (1:9 dilution) was prepared in HEPES buffer ( 25 mM HEPES, 1 mM EDTA, 1 mM benzamidine, 1 mM 4-(2-aminoethyl)-benzene+sulfonyl fluoride (AEBSF), 1 μM leupeptin, 1 μM pepstatin, 1 μM aprotinin, pH 7.5). Proteins of these homogenates were separated by SDS-PAGE using 10% resolving gels (Tris-HCl ready gels, BIO-RAD, Hercules, Calif.). Proteins were transferred from the gel to a nitrocellulose membrane at 100 volts for 60 min. The membranes were blocked with 3% BSA in 139 mM NaCl, 2.7 mM KH₂PO₄, 9.9 mM Na₂HPO₄, and 0.05% Tween-20 (PBST) and 1% sodium azide. After two 5 minutes washes in 139 mM NaCl, 2.7 mM KH₂PO₄, 9.9 mM Na₂HPO₄ (PBS) , membranes were incubated with GLUT4 polyclonal antibody RaIRGT, Biogenesis, Sandown, N H) for 1 hour at room temperature. After two 5 minutes washes in PBST and two 5 minutes washes in PBS, membranes were exposed to horseradish peroxidase-conjugated donkey anti-rabbit IgG (Amersham Life Science, Arlington Heights, Ill.) for 1 hour at room temperature. After washing twice with PBST and twice with PBS the membranes were incubated in enhanced chemoluminescence detection reagent and then visualized on enhanced chemoluminescence hyperflim (Amersham Life Sciences). Relative amounts of GLUT4 were then quantified using a Hewlett Packard ScanJet 6200C and SigmaGel software (SPSS, Inc, Chicago, Ill.). Total intensity of GLUT4 spots on the developed hyperfilm was expressed as a fraction of intensity shown by a GLUT4 standard run on the same gel. The GLUT4 standard was a plasma membrane fraction prepared as described previously. Kurth-Kraczek, E. J. et al. Diabetes 48 (1999).

Hexokinase activity was determined spectrophotometrically at 30 C on 700×g supernatants of the same homogenate as was used for GLUT4 measurement Uyeda, K. & E. Racker, J Biol Chem 240:4682-4688 (1965).

Results are expressed as means ±SEM. Statistically significant differences between control and AICAR treated rats were determined using Student's t test.

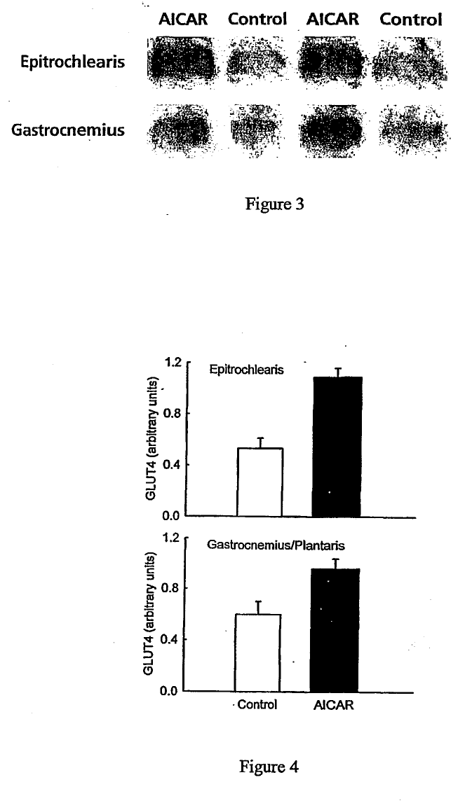

FIGS. 3 and 4 show marked increases in GLUT4 in both epitrochlearis and in gastrocnemius/plantaris in response to injection of rats with AICAR for five days. Western blots of total GLUT4 protein in epitrochlearis and gastrocnemius/plantaris muscles from two rats injected with AICAR (1 mg/g body weight) and from two rats injected with saline for 5 days are shown are shown in FIG. 3. While the relative GLUT4 levels in epitrochlearis of rats treated for 5 days with AICAR (1 mg/g/d) are shown in FIG. 4. Relative total intensity of GLUT4 from muscles from AICAR and saline-injected rats is expressed as a fraction of intensity of standard GLUT4 spots run on all gels. Values for GLUT4 in AICAR injected rats are significantly different from controls, p<0.001 for epitrochlearis and p<0.01 for gastrocnemius (n=10-12/group). We noted also in preliminary experiments that larger rats (weighing 350-450 grams) responded to AICAR injections (0.5 mg/g body weight) with a significant increase (0.76±0.08 vs 0.32±0.05 arbitrary units, p<0.02, n=5/group) in total GLUT4.

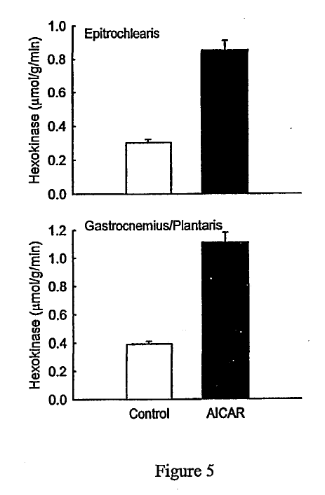

The effect of 5 days of AICAR injections (1 mg/g/d) on hexokinase activity in epitrochlearis and gastrocnemius/plantaris muscles is illustrated in FIG. 5. Values for AICAR injected rats are significantly different from controls, (n=10-11/group). Hexokinase activity increased markedly in response to five days of AICAR injections. The increase was approximately 2.8 fold over control values in both epitrochlearis and in the gastrocnemius/plantaris. Both increases were highly significant, p<0.001.

In rats killed 24 hrs following the last of five daily injections of AICAR, gastrocnemius/plantaris glycogen was 87±4 μmol glucose units/g compared to 43±2 μmol glucose units/g. This difference was highly significant (p<0.001).

It is well documented that concurrent with an increase in GLUT4, an increase in muscle hexokinase activity is also seen in response to endurance exercise training. Holloszy, J. O. & P. A. Hansen 128:99-193 (1996); Ivy, J. L. Sports Med. 24:321-336 (1997). The finding of increased hexokinase activity with chronic activation of AMPK with AICAR in sedentary rats, lends credence to the idea that repetitive AMPK activation is mediating the effect of chronic muscle contraction on these training adaptations.

Glycogen supercompensation is another well-established effect of endurance exercise training that appears to occur concurrently with an increase in muscle GLUT4. Host, H. H. et al. J. Appl. Physiol. 85:133-138 (1998). Although there are other factors that may be responsible for the elevated glycogen in muscles of the rats chronically treated with AICAR (but killed 1 day after the last injection), the increased GLUT4 in the muscle may allow increased glucose uptake (after the acute effects of AICAR are gone) and the accumulation of more than double the amount of glycogen seen in the saline-injected controls.

Relatively few agents have been described which are effective in manipulating GLUT4 levels in muscle. Ezaki, O. Biochem. Biophys. Res. Comm. 241:16 (1997); Tsunoda, N. et al. Biochem. Biophys. Res. Comm. 239:503-509 (1997). The data from this study (showing inactivation of ACC, decrease in malonyl-CoA, and increase in total GLUT4) suggest the possibility of targeting the AMPK signaling system for treatment of insulin resistance. This can be done naturally with exercise, but for those who are unable to exercise, pharmacologic manipulation of this signaling system is feasible. At least some patients with type 2 diabetes respond to an acute bout of exercise with translocation of GLUT4 to plasma membranes. Kennedy, J. W. et al. Diabetes 48:1192-1197 (1999). It now appears that chronic periodic activation of AMPK with chemical activators is useful in manipulating GLUT4. In addition, these studies may provide the rationale for searching for possible defects in the AMPK signaling system as a cause of insulin resistance and dyslipidemia in some forms of type 2 diabetes.

Summary

In summary, chronic activation of skeletal muscle AMPK by injection of AICAR into sedentary rats results in significant increases in total GLUT4 and hexokinase activity, similarly to the changes induced by endurance exercise training. Previous studies have demonstrated that muscle contraction occurring during exercise or in response to electrical stimulation increase AMPK activity. The increases in skeletal muscle GLUT4 and hexokinase induced by training are mediated by AMPK activation. Because exercise training has been shown to be an effective treatment for type 2 diabetes and insuling resistance, AMPK activators can be administered artificially stimulate AMPK and provide a patient with the benefits of exercise.

The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope. All patents, publications, and commercial materials cited herein are hereby incorporated by reference.

1. A method for treating diabetes in a mammal comprising:
administering a therapeutically effect amount of an AMP-activated protein kinase activator.

2. The method of claim 1, wherein administering a therapeutically effective amount of an AMP-activated protein kinase activator results in an increase in GLUT4 in muscle of the mammal.

3. The method of claim 1, wherein administering a therapeutically effective amount of an AMP-activated protein kinase activator induces translocation of GLUT4 to the membrane surface of the muscle.

5. The method of claim 1, wherein the AMP-activated protein kinase activator comprises 5-aminoimidazole-4-carboxamide-riboside.

6. The method of claim 1, wherein the AMP-activated protein kinase activator is subcutaneously injected into the mammal.

7. The method of claim 1, wherein the AMP-activated protein kinase activator comprises an AMP analogue.

8. The method of claim 7, wherein the AMP analogue is selected from the group consisting of adenosine-5′-thiomonophosphate, adenosine 5′-phosphoramidate, formycin A 5′-monophosphate, and ZMP.

9. The method of claim 7, wherein the AMP analogue is modified previous to administration to facilitate uptake by cells.

10. The method of claim 7, wherein the AMP analogue is administered intra-cellularly.

11. The method of claim 7, wherein the AMP analogue comprises 5-aminoimidazole-4-carboxamide ribonucleoside.

12. The method of claim 11, wherein 5-aminoimidazole-4-carboxamide ribonucleoside is administered at a dose from about 0.5 to at least about 1.0 mg/g body weight.

13. The method of claim 1, wherein the AMP-activated protein kinase activator is administered acutely.

14. The method of claim 1, wherein the AMP-activated protein kinase activator is administered chronically.

15. The method of claim 1, wherein the AMP-activated protein kinase activator is administered intermittently.

16. A method for treating insulin resistance in a mammal comprising:
administering a therapeutically effect amount of an AMP-activated protein kinase activator.

17. The method of claim 16, wherein administering a therapeutically effective amount of an AMP-activated protein kinase activator results in an increase in GLUT4 in muscle of the mammal.

18. The method of claim 16, wherein administering a therapeutically effective amount of an AMP-activated protein kinase activator induces translocation of GLUT4 to the membrane surface of the muscle.

19. The method of claim 16, wherein the AMP-activated protein kinase activator comprises 5-aminoimidazole-4-carboxamide-riboside.

20. The method of claim 16, wherein the AMP-activated protein kinase activator is subcutaneously injected into the mammal.

22. The method of claim 16, wherein the AMP-activated protein kinase activator comprises an AMP analogue.

23. The method of claim 22, wherein the AMP analogue is selected from the group consisting of adenosine-5′-thiomonophosphate, adenosine 5′-phosphoramidate, formycin A 5′-monophosphate, and ZMP.

24. The method of claim 22, wherein the AMP analogue is modified previous to administration to facilitate uptake by cells.

25. The method of claim 22, wherein the AMP analogue is administered intra-cellularly.

26. The method of claim 22, wherein the AMP analogue comprises 5-aminoimidazole-4-carboxamide ribonucleoside.

27. The method of claim 26, wherein 5-aminoimidazole-4-carboxamide ribonucleoside is administered at a dose from about 0.5 to at least about 1.0 mg/g body weight.

28. The method of claim 16, wherein the AMP-activated protein kinase activator is administered acutely.

29. The method of claim 16, wherein the AMP-activated protein kinase activator is administered chronically.

30. The method of claim 16, wherein the AMP-activated protein kinase activator is administered intermittently.
